(19) United States
(12) Patent Application Publication
(10) Pub. No.: US 2022/0000424 A1
(43) Pub. Date: Jan. 6, 2022

(54) GARMENT, MEASUREMENT APPARATUS AND MONITORING SYSTEM

(71) Applicant: POSH WELLNESS LABORATORY INC., Tokyo (JP)

(72) Inventor: Satoru Nebuya, Tokyo (JP)

(21) Appl. No.: 17/376,347

(22) Filed: Jul. 15, 2021

Related U.S. Application Data
(63) Continuation of application No. PCT/JP2019/001101, filed on Jan. 16, 2019.

(51) Int. Cl. A61B 5/00; A61B 5/11; A61B 5/0205; A41D 13/12

(52) U.S. Cl. CPC A61B 5/6804; A61B 5/1126; A61B 5/0809; A41D 13/1281; A61B 2562/0219; A61B 5/0205

(57) ABSTRACT

A garment worn by a user includes a sensor material that includes a) an insulating material stretchable in a longitudinal direction and b) a conductive fiber having conductivity and coiled around the insulating material, and a plurality of connecting portions electrically connected to three or more portions of the conductive fiber, wherein at least a portion of the conductive fiber includes a fixing region fixed such that the fixing region does not stretch or shrink even when the insulating material stretches and shrinks, and at least two connecting portions of the plurality of connecting portions are electrically connected to each of two different portions of the fixing region.

### CROSS-REFERENCE TO RELATED APPLICATIONS

The present application is a continuation application of International Application number PCT/JP2019/001101, filed on Jan. 16, 2019. The contents of this application are incorporated herein by reference in their entirety.

### BACKGROUND OF THE INVENTION

Conventionally, a measurement apparatus that easily detects movement, breathing, a heartbeat, and the like of a person has been desired. In particular, there has been a need for a system that can measure and monitor the following: a) breathing motion during sleep to determine whether or not apnea happens, b) sudden death of a patient in a hospital or the like, c) infant death due to a prone position, and the like. As such a system, a system that detects breathing motion and the like of a person by using a bed or the like equipped with a sensor, a system that detects movement of a person by using laser radiation or an imaging device, and the like have been known, for example.

However, when states of a plurality of users are monitored using beds with sensors, a plurality of beds with sensors must be prepared for the number of users to be monitored, and it has been difficult to introduce such a large and expensive system. In addition, when the user is optically monitored, detection sensitivity may decrease when the user enters a blind spot of an optical system. Therefore, it has been difficult to realize a system that detects and monitors the movement, breathing, heartbeat, and the like of a person with high accuracy and ease.

### BRIEF SUMMARY OF THE INVENTION

The present disclosure focuses on these points, and an object of the present disclosure is to easily detect the movement, breathing, heartbeat, and the like of a person.

The first aspect of the present disclosure provides a garment worn by a user, that includes a sensor material that includes a) an insulating material stretchable in a longitudinal direction and b) a conductive fiber having conductivity and coiled around the insulating material, and a plurality of connecting portions electrically connected to three or more portions of the conductive fiber, wherein at least a portion of the conductive fiber includes a fixing region fixed such that the fixing region does not stretch or shrink even when the insulating material stretches and shrinks, and at least two connecting portions of the plurality of connecting portions are electrically connected to each of two different portions of the fixing region.

The second aspect of the present disclosure provides a measurement apparatus for measuring a state of a user wearing a garment, wherein the garment includes a sensor material that includes a) an insulating material stretchable in a longitudinal direction and b) a conductive fiber having conductivity and coiled around the insulating material, and a plurality of connecting portions electrically connected to three or more portions of the conductive fiber, the measurement apparatus includes a measurement part that measures impedance between two different connecting portions of the plurality of connecting portions of the garment, a switching part that switches an electrical connection between the plurality of connecting portions and the measurement portion, a controller that controls the switching part such that impedances between two adjacent connecting portions are measured by the measurement part, and an identification part that identifies a state of the user wearing the garment on the basis of the measurement result of the impedance.

The third aspect of the present disclosure provides a monitoring system that includes a garment, and a measurement apparatus that measures a state of a user wearing the garment, wherein the monitoring system monitors a state of the user, the garment includes a sensor material that includes a) an insulating material stretchable in a longitudinal direction and b) a conductive fiber having conductivity and coiled around the insulating material, and a plurality of connecting portions electrically connected to three or more portions of the conductive fiber, the measurement apparatus includes a measurement part that measures impedance between two different connecting portions of the plurality of connecting portions of the garment, a switching part that switches an electrical connection between the plurality of connecting portions and the measurement portion, a controller that controls the switching part such that impedances between two adjacent connecting portions are measured by the measurement part, and an identification part that identifies a state of the user wearing the garment on the basis of the measurement result of the impedance, wherein the monitoring system further includes an acquisition part that is connected to each of the plurality of measurement apparatuses and acquires states of the plurality of users respectively wearing the plurality of garments, and a detection part that detects an abnormal state among the states of the plurality of users.

### BRIEF DESCRIPTION OF THE DRAWINGS

FIG. 1 shows a configuration example of a monitoring system 10 according to the present embodiment.

FIG. 2 shows a configuration example of a garment 20 and a measurement apparatus 30 according to the present embodiment.

FIG. 3 shows a configuration example of a sensor element 100 according to the present embodiment.

FIG. 4 shows a configuration example of a sensor material 120 according to the present embodiment.

FIG. 5 shows a variation of the garment 20 and the measurement apparatus 30 according to the present embodiment.

### DETAILED DESCRIPTION OF THE INVENTION

Hereinafter, the present invention will be described through exemplary embodiments of the present invention, but the following exemplary embodiments do not limit the invention according to the claims, and not all of the combinations of features described in the exemplary embodiments are necessarily essential to the solution means of the invention.

### <Configuration Example of Monitoring System 10>

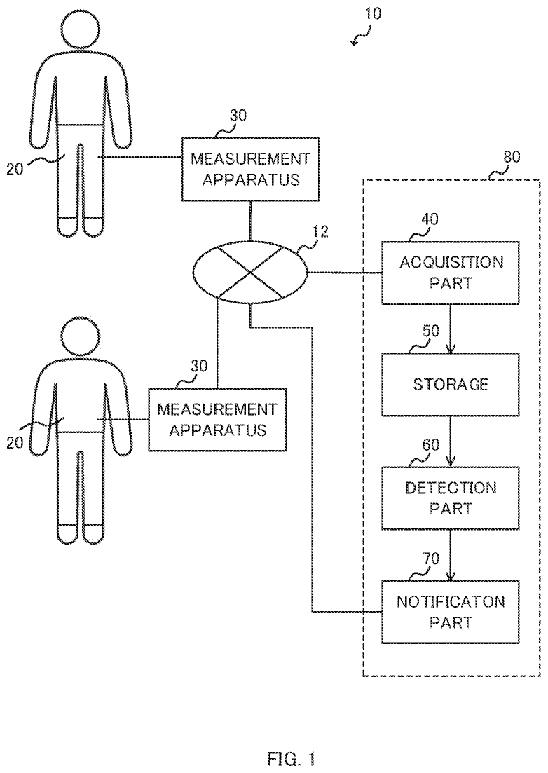

FIG. 1 shows a configuration example of a monitoring system 10 according to the present embodiment. The monitoring system 10 monitors abnormal movement and the like of a user on the basis of a detection signal of a sensor provided to a garment worn by the user. The monitoring system 10 includes a garment 20, a measurement apparatus 30, an acquisition part 40, a storage 50, a detection part 60, and a notification part 70.

The garment 20 is worn by the user, and is provided with a sensor. The garment 20 is underwear, pajamas, a hospital gown, room wear, and the like, for example. The sensor provided to the garment 20 will be described later.

The measurement apparatus 30 measures a state of the user wearing the garment 20 on the basis of a result of the detection by the sensor. The measurement apparatus 30 measures, for example, states of movement, breathing, a heartbeat, and the like of the user. The measurement apparatus 30 may be attached to the garment 20 of the user, or alternatively, may be provided separately from the garment 20. The garment 20 and the measurement apparatus 30 are connected by wire or wirelessly, for example. In the monitoring system 10, such a set of the garment 20 and the measurement apparatus 30 is provided for each user, for example. The measurement apparatus 30 transmits a result of the measurement to a server 80 via a network 12, for example. It should be noted that the network 12 is the Internet, for example, but may be a local area network instead.

The server 80 includes the acquisition part 40, the storage 50, the detection part 60, and the notification part 70.

The acquisition part 40 is connected to each of a plurality of measurement apparatuses 30, and acquires states of the plurality of users wearing the plurality of garments 20. The acquisition part 40 acquires the measurement results of the measurement apparatuses 30 indicating the users' states. The acquisition part 40 may be connected to the plurality of measurement apparatuses 30 via the network 12, or may be directly connected to the plurality of measurement apparatuses 30.

The storage 50 stores the acquired measurement results of the plurality of measurement apparatuses 30. Further, the storage 50 may store intermediate data, calculation results, threshold values, parameters, and the like generated (or used) by the monitoring system 10 in the course of operation, respectively. Furthermore, in response to a request from each part in the monitoring system 10, the storage 50 may supply the stored data to the requester.

The detection part 60 detects an abnormal state from among the plurality of users' states. The detection part 60 detects a user in an apneic state among the plurality of users, for example. In this case, the detection part 60 may detect the duration and the like of the apnea of the user. In addition, the detection part 60 detects a user whose heartbeat is less than a threshold value among the plurality of users, for example. In this case, the detection part 60 may detect the duration and the like of the state where the user's heartbeat is less than the threshold value.

When the detection part 60 detects a user in the abnormal state, the notification part 70 notifies the outside that the user in the abnormal state has been detected. The notification part 70 displays, for example, the fact that an abnormality has been detected on a display part or the like for an operator or the like of the monitoring system 10. The notification part 70 may notify that the abnormality has been detected by generating sound or the like. Further, the notification part 70 may notify an external server or the like that the abnormality has been detected via the network 12.

As described above, since the monitoring system 10 according to the present embodiment measures the user's state on the basis of the result of the detection by the sensor provided to the user's garment 20, no large-scale measurement apparatus or the like using a bed and the like is required. For example, the present disclosure can be easily applied to a plurality of users hospitalized in a relatively large hospital or the like. Further, since the monitoring system 10 can monitor the plurality of users by using the network 12 or the like, the monitoring system 10 can be configured without being bound by specific buildings, regions, and the like. The monitoring system 10 can monitor the states of the plurality of users, including users undergoing home treatment, for example. The following is a description of the garment 20 and the measurement apparatus 30 used by the monitoring system 10.

### <Configuration Example of Garment 20 and Measurement Apparatus 30>

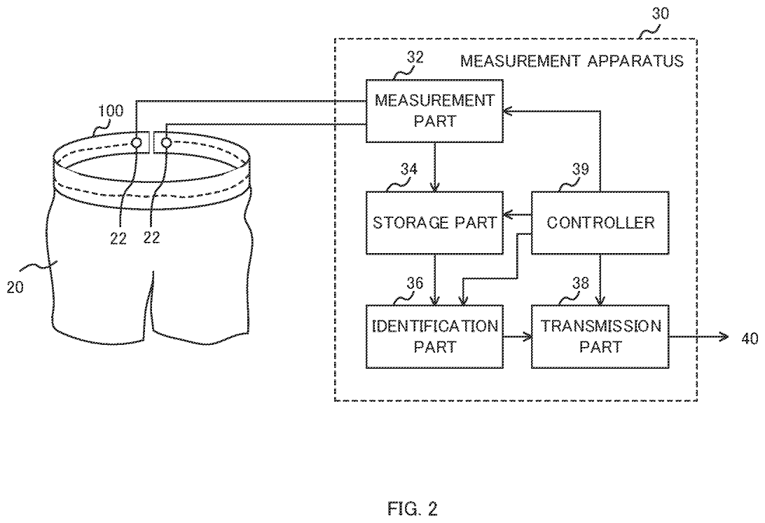

FIG. 2 shows a configuration example of the garment 20 and the measurement apparatus 30 according to the present embodiment. The garment 20 is provided with a sensor element 100 as a sensor for detecting the state of the user. The sensor element 100 is provided such that the sensor element stretches and shrinks in response to movement of the user's body. The sensor element 100 is an element whose impedance changes according to the movement of the user's body, for example. FIG. 2 shows an example in which the sensor element 100 is provided such that the sensor element 100 is in close contact with a user's abdominal circumference, and the impedance of the sensor element 100 changes according to the displacement of the user's abdominal circumference. It should be noted that, in the present embodiment, an example in which the inductance of the sensor element 100 changes in accordance with the movement of the user's body will be described. Such a sensor element 100 will be described later.

The garment 20 includes a plurality of connecting portions 22 connected to the measurement apparatus 30. Each of the connecting portions 22 is electrically connected to the sensor element 100. The plurality of connecting portions 22 function as input/output terminals of the sensor element 100. For example, among the plurality of connecting portions 22, one or more connecting portions 22 are portions of buttons provided to the garment 20. FIG. 2 shows an example in which the connecting portions 22 are provided at both ends of the sensor element 100.

The button is a metal fastener such as a snap button, for example. In this case, it is desirable that the snap button includes a set of a detachable concave button and convex button, and either one of the concave button and convex button is sewn to the garment 20. Thus, the plurality of connecting portions 22 can be easily attached to the garment 20. Further, the user can wear the garment 20 provided with the connecting portions 22 without feeling uncomfortable. In addition, one of the concave button and the convex button can be used as a terminal for connecting the garment 20 to a cable that electrically connects the garment 20 and the measurement apparatus 30.

The measurement apparatus 30 includes a measurement part 32, a storage part 34, an identification part 36, a transmission part 38, and a controller 39. The measurement part 32 measures the inductance between two different connecting portions 22 among the plurality of connecting portions 22 of the garment 20. FIG. 2 shows an example in which the measurement part 32 measures the inductance between two connecting portions 22 provided at respective ends of the sensor element 100.

The measurement part 32 may measure the inductance using a known measurement method, and is not described in detail here. For example, the measurement part 32 supplies an AC signal having a predetermined amplitude voltage to the sensor element 100, and measures the inductance on the basis of a result of measuring an AC current flowing through the sensor element 100. It is desirable that the measurement part 32 continuously measures the inductance to measure a change in the inductance. In this case, the measurement part 32 may measure the inductance every predetermined time, or alternatively, may continue to measure the inductance at substantially constant time intervals.

The storage part 34 stores a value of the inductance measured by the measurement part 32. Further, the storage part 34 may store intermediate data, calculation results, threshold values, parameters, and the like generated (or used) by the measurement apparatus 30 in the course of operation, respectively. Furthermore, in response to a request from each part in the monitoring system 10, the storage part 34 may supply the stored data to the requester.

The identification part 36 identifies the state of the user wearing the garment 20 on the basis of a result of measuring the inductance by the measurement part 32. The identification part 36 identifies the user's state in accordance with a temporal change of the inductance, for example. For example, if the change of the inductance has no periodicity and the inductance temporarily changes more than a threshold value, the identification part 36 identifies that the user is in a moving state such as a turning over in bed. Further, the identification part 36 may identify that the user is in an apneic state if the periodic change of the inductance is less than the threshold value. Furthermore, if the inductance changes at a substantially constant period and a range of the change of the inductance is within predetermined upper and lower limits, the identification part 36 may identify that the user's respiration rate is the reciprocal of said period.

Moreover, if the temporal change of the inductance changes at a substantially constant period and a range of the change of the amplitude value is within predetermined upper and lower its, the identification part 36 may identify that the user's heartbeat is the reciprocal of the period. Alternatively or additionally, the identification part 36 may identify the user's state by comparing a) a measured change pattern of the inductance with b) a change pattern of the inductance that changes in accordance with the breathing of the user stored in the storage part 34 in the past.

The transmission part 38 transmits the result identified by the identification part 36 to the acquisition part 40. When the measurement apparatus 30 and the acquisition part 40 are connected wirelessly, the transmission part 38 includes a transmission/reception circuit such as an antenna, and transmits a result of the identification to the acquisition part 40 wirelessly.

The controller 39 controls operations of the measurement part 32, the storage part 34, the identification part 36, and the transmission part 38. The controller 39 controls, for example, the timing at which the measurement part 32 measures the inductance, the timing at which the storage part 34 stores the measurement result, the timing at which the identification part 36 acquires the measurement result and identifies the user's state, and the timing at which the transmission part 38 transmits the identification result. The controller 39 controls, for example, each part to measure the inductance at a predetermined time or time interval, and transmits the identification result to the monitoring system 10. The controller 39 is a Central Processing Unit (CPU), for example.

The measurement apparatus 30 according to the present embodiment is preferably formed of an integrated circuit or the like. Further, the measurement apparatus 30 is more preferably configured as a mobile device with a battery or the like. This allows the measurement apparatus 30 to be easily held in a pocket, belt, bag, or the like of the user wearing the garment 20. Further, the measurement apparatus 30 may be configured as a portion of a terminal carried by the user. The above-described measurement apparatus 30 measures the user's state on the basis of the change of the inductance of the sensor element 100 provided to the garment 20. Such a sensor element 100 whose inductance changes according to the user's state will be described below.

### <Configuration Example of Sensor Element 100>

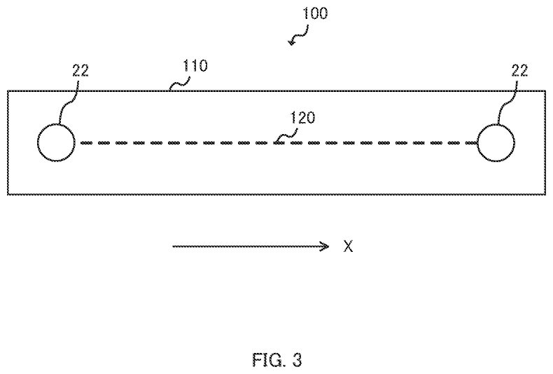

FIG. 3 shows a configuration example of the sensor element 100 according to the present embodiment. FIG. 3 shows an example in which the sensor element 100 functions as an elastic cord. The sensor element 100 includes a base 110 and a sensor material 120. It should be noted that the sensor element 100 shown in FIG. 3 is an example of a sensor element further including connecting portions 22.

The base 110 is formed as a string or strip using a thread that can stretch and shrink in a longitudinal direction. The base 110 is a band, string, cloth, or the like made of an elastic material, for example. Further, the base 110 may be partially made of an elastic material. FIG. 3 shows an example in which the base 110 is a belt-like elastic cord, and stretches and shrinks in the X direction.

The sensor material 120 is formed to be stretched and shrunk in the longitudinal direction. The sensor material 120 is coupled to the base 110 such that the base 110 can be stretched and shrunk in a direction in which the sensor material 120 stretches and shrinks. The sensor material 120 may be woven into the base 110, for example, or alternatively, may be sewn to the base 110. Further, the sensor material 120 may be fixed to the base 110 by using an adhesive or the like. Furthermore, the sensor material 120 may be fixed by being sewn to the base 110 together with the connecting portions 22. As a result, for example, when the base 110 stretches and shrinks in the longitudinal direction, the sensor material 120 stretches and shrinks in the longitudinal direction in the same way as the base 110.

Thus, the sensor material 120 includes an inductance component whose inductance value changes as the sensor material 120 stretches and shrinks in the longitudinal direction. That is, the sensor material 120 is coupled to the base 110 such that, the inductance value of the sensor material 120 changes when the base 110 stretches and shrinks in the X direction.

Further, the connecting portion 22 is provided to the base 110 such that the sensor material 120 is electrically connected to the connecting portion 22. FIG. 3 shows an example in which two connecting portions 22 are provided to the base 110 and are electrically connected to respective ends of the sensor material 120. In this case, by connecting the two connecting portions 22 and the measurement apparatus 30, the measurement apparatus 30 can measure the inductance of the sensor material 120 between the two connecting portions 22. Such a sensor material 120 will be described below.

### <Configuration Example of Sensor Material 120>

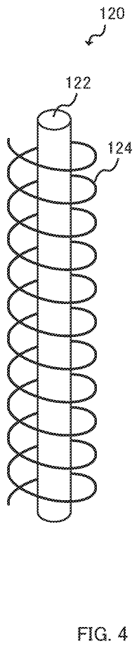

FIG. 4 shows a configuration example of the sensor material 120 according to the present embodiment. The sensor material 120 includes an insulating material 122 and a conductive fiber 124. The insulating material 122 can stretch and shrink in the longitudinal direction. The insulating material 122 is made of an elastic material. The insulating material 122 is made of rubber, polymer, or the like, for example. The insulating material 122 may include a mated al having a high dielectric constant. The insulating material 122 is formed as a thread extending in the longitudinal direction, for example.

The conductive fiber 124 is made of a material having conductivity. Further, the conductive fiber 124 may be formed by attaching a conductive material to a fibrous material. The conductive fiber 124 may be a conductive film or the like, for example, or alternatively, may be formed by attaching conductive ink or the like to a thread-like rubber, polymer, or the like. The conductive fiber 124 is coiled around the insulating material 122.

The above-described sensor material 120 has an inductance component corresponding to the number of turns, diameter, and length of the conductive fiber 124, the dielectric constant of the insulating material 122, and the like. Further, the inductance value of the sensor material 120 increases and decreases as the conductive fiber 124 stretches and shrinks in the longitudinal direction. It should be noted that the connecting portions 22 are electrically connected to at least two portions of the conductive fiber 124. For example, when the two connecting portions 22 are connected to the conductive fiber 124, the measurement part 32 can measure the value of the inductance of the conductive fiber 124 connected between the two connecting portions 22.

Here, the conductive fiber 124 is preferably fixed to a plurality of different portions of the insulating material 122. For example, the conductive fiber 124 is fixed, by using an adhesive or the like, to the insulating material 122 at a) a first position and b) a position distanced from the first position by the length of the conductive fiber 124 in a state where the conductive fiber 124 is not stretched or shrunk. Thus, the conductive fiber 124 stretches and shrinks integrally with the insulating material 122. That is, the sensor material 120 functions as a sensor whose inductance value changes in accordance with the stretching and shrinking of the sensor material 120 in the longitudinal direction. When such a sensor material 120 is coupled to the base 110, the conductive fiber 124 stretches and shrinks integrally with the base 110. That is, the sensor material 120 functions as a sensor for detecting the length of the base 110 in the longitudinal direction.

Alternatively or additionally, said sensor material 120 may be coupled to the base 110 such that the conductive fiber 124 is coupled to different portions of the base 110. Also in this case, since the conductive fiber 124 integrally stretches and shrinks with the base 110, the sensor material 120 functions as a sensor for detecting the length of the base 110 in the longitudinal direction.

When the sensor element 100 to which the above sensor material 120 is coupled is used as a part of the garment 20, the sensor element 100 can detect the displacement of the body of the user wearing the garment 20. For example, by using the sensor element 100 as an elastic cord of the garment 20 such as a shirt, underwear, pants, or the like, the sensor element 100 can be arranged to surround the neck, chest, abdomen, or waist of the user. As a result, the sensor element 100 stretches and shrinks in the longitudinal direction in accordance with the displacement of the user's body caused by the breathing, heartbeat, and the like of the user, and the inductance of the sensor element 100 is changed according to the user's state. Accordingly, the measurement apparatus 30 can measure the user's state by measuring the inductance of the sensor element 100.

As described above, the sensor material 120 according to the present embodiment is coupled to an elastic cord, an elastic cloth, or the like, and works as the sensor element 100 used as a part of the garment 20. Since such a sensor material 120 is formed as a thread as described with reference to FIG. 4, it is easy to form the sensor element 100, and it is also easy to attach the sensor element 100 to the garment 20 and replace it.

In addition, since the sensor element 100 can be fixed such that the sensor element 100 surrounds the user's body as a stretchable elastic cord, the deviation of the sensor element 100 caused by the user's movement and turning over in bed hardly occurs. Further, even if deviation occurs in the arrangement of the sensor element 100, reduction in the detection sensitivity can be prevented because the sensor element 100 is deviated while being fixed around the user's body. Furthermore, since the measurement apparatus 30 detects the periodic changes in the inductance, the noise components caused by aperiodic movement or the like of the user can be easily removed. Accordingly, the measurement apparatus 30 can easily detect the movement, breathing, heartbeat, and the like of a person. As a result, the cost can be reduced, and a simple and highly accurate monitoring system 10 can be realized.

An example in which the sensor material 120 according to the present embodiment is coupled to the base 110 to be provided on the garment 20 has been described above, but the present disclosure is not limited thereto. The sensor material 120 may be provided on the garment 20. For example, when the garment 20 is formed using a cloth or the like having elasticity at least in part, the sensor material 120 may be coupled to said cloth having elasticity. In this case, it is desirable that the cloth can stretch and shrink in a direction in which the sensor material 120 stretches and shrinks.

For example, when an elastic cord is already provided to the garment 20, the sensor material 120 may be coupled to said elastic cord. Further, when the garment 20 is made of a cloth that stretches and shrinks while being in close contact with the user's body, such as tights, spats, rash guards, stomach wraps, wrist bands, and the like, the sensor material 120 may be coupled to said cloth.

Thus, as long as the displacement of the user wearing the garment 20 can be detected, the sensor material 120 or the sensor element 100 can be placed in any part of the garment 20. For example, the sensor element 100 may be at least a portion of a belt of a gown and the like. Further, the sensor element 100 may be placed on a chest, neck, arm, wrist, or the like. As long as the displacement of the user can be detected, the sensor element 100 may be provided to a portion other than the garment 20. For example, the sensor element 100 may be at least a portion of an elastic cord of headgear. The sensor element 100 may be a portion of a belt, a suspender, or the like.

An example in which the measurement apparatus 30 according to the present embodiment measures the inductance between the two different portions of the conductive fiber 124 has been described above. For the purpose of further improving the measurement sensitivity, it is desirable that the sensor material 120 is longer, and it is more desirable that the sensor material 120 is provided on the garment 20 such that the sensor material 120 surrounds the user's body. The measurement apparatus 30 may measure the inductances at a plurality of different portions of the conductive fiber 124. Such a measurement apparatus 30 will be described below.

### <Example of Measuring Inductances at a Plurality of Portions of Conductive Fiber 124>

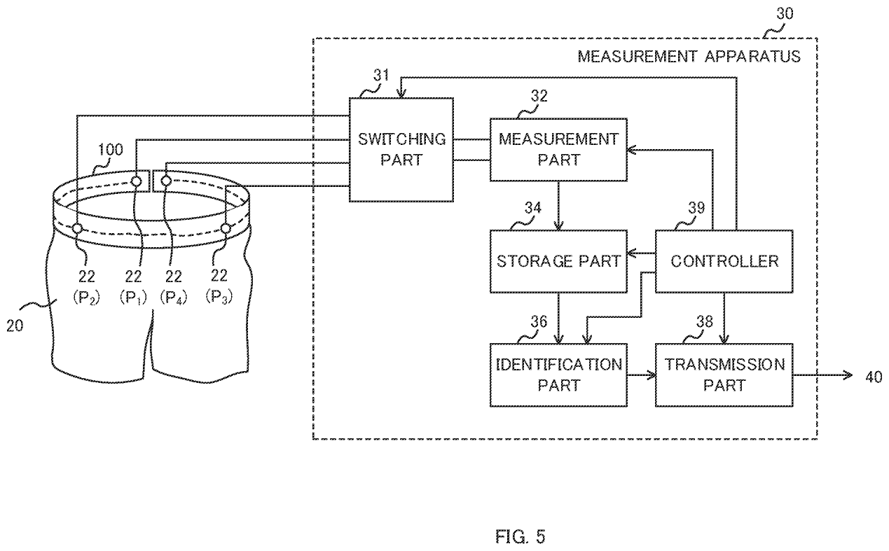

FIG. 5 shows a variation of the garment 20 and the measurement apparatus 30 according to the present embodiment. The garment 20 according to the variation has a plurality of connecting portions 22 electrically connected to each of three or more portions of the conductive fiber 124. The plurality of connecting portions 22 are respectively connected to a plurality of portions spaced at predetermined intervals in the longitudinal direction of the conductive fiber 124, for example. FIG. 5 shows an example in which the plurality of connecting portions 22 are connected to the plurality of portions spaced at equal intervals in the longitudinal direction of the conductive fiber 124. In FIG. 5, the portions of the conductive fibers 124 are shown as P₁ to P₄.

The measurement apparatus 30 according to the variation further includes a switching part 31 for switching the electrical connection between the plurality of connecting portions 22 and the measurement part 32. The switching part 31 includes a plurality of switches. The controller 39 supplies, to the switching part 31, a control signal for switching the plurality of switches of the switching part 31, and controls the switching part 31 such that the measurement part 32 can measure the inductances of the plurality of different portions of the conductive fiber 124. For example, the controller 39 controls the switching part 31 to cause the measurement part 32 to measure the inductance between two adjacent connecting portions 22.

In the case of the example of FIG. 5, the controller 39 controls the switching part 31 to a) electrically connect the measurement part 32 to the connecting portions 22 connected to P₁ and P₂ of the conductive fiber 124, for example, and b) electrically disconnect the measurement part 32 from the connecting portions 22 connected to P₃ and P₄ of the conductive fiber 124. Thus, the measurement part 32 can measure the inductance between P₁ and P₂ of the conductive fiber 124.

In this case, the controller 39 then controls the switching part 31 to a) electrically connect the measurement part 32 to the connecting portions 22 connected to P₂ and P₃ of the conductive fiber 124 and b) electrically disconnect the measurement part 32 from the connecting portions 22 connected to P₁ and P₄ of the conductive fiber 124. Thus, the measurement part 32 can measure the inductance between P₂ and P₃ of the conductive fiber 124. Similarly, the controller 39 may control the switching part 31 to cause the measurement part 32 to measure the inductance between P₃ and P₄.

As described above, the measurement apparatus 30 according to the variation can measure the user's state in more detail by measuring the inductances of the plurality of different portions of the conductive fiber 124. For example, the measurement apparatus 30 may identify the state of the user's heartbeat on the basis of the measurement result of the portion of the conductive fiber 124 closer to the user's heart, and may identify the state of the user's breathing on the basis of the measurement result of the portion of the conductive fiber 124 closer to the user's lung.

In the case of the example of FIG. 5, the measurement apparatus 30 can measure each of a) a displacement on the right side of the user's abdomen from a change in the inductance between P₁ and P₂, b) a displacement on the center of the user's abdomen from a change in the inductance between P₂ and P₃, and c) a displacement on the left side of the user's abdomen from a change in the inductance between P₃ and P₄. Accordingly, the measurement apparatus 30 can identify the state of the user's movement such as walking or lying back from the difference in displacements between the left side of the abdomen and the right side of the abdomen of the user, for example. Further, the measurement apparatus 30 may identify the state of the user's breathing and/or heartbeat from the difference between each of the displacements of the left side of the abdomen and the right side of the abdomen of the user and the displacement of the center of the abdomen of the user.

It should be noted that an example in which the measurement apparatus 30 according to the present variation measures the inductances of the plurality of different portions of one conductive fiber 124 has been described, but the present disclosure is not limited thereto. The measurement apparatus 30 may measure the inductances of the plurality of conductive fibers 124. In this case, the garment 20 is provided with a plurality of sensor elements 100, for example.

Further, when the plurality of conductive fibers 124 are provided to the garment 20, the measurement apparatus 30 may measure the inductances of the plurality of different portions of each of the conductive fibers 124. Also in this case, the controller 39 controls the switching part 31 such that the measurement part 32 can measure a portion of the conductive fiber 124 to be measured. Furthermore, the measurement apparatus 30 may include a plurality of measurement parts 32 to measure the inductances in parallel.

### <Example of Measuring Inductance Using a Reference Value>

An example in which the measurement apparatus 30 according to the present embodiment measures the inductance of the conductive fiber 124 has been described above. Here, the measurement apparatus 30 may measure the inductance of the conductive fiber 124 while comparing the inductance of the conductive fiber 124 with the inductance of a reference portion provided to the conductive fiber 124. In this case, at least a portion of the conductive fiber 124 has a fixing region fixed such that the conductive fiber 124 does not stretch or shrink even when the insulating material 122 stretches and shrinks. That is, the fixing region of the conductive fiber 124 is a portion that is fixed such that the conductive fiber 124 maintains a certain length in the longitudinal direction, independent of the user's state. The fixing region of the conductive fiber 124 may be sewn to the garment 20 or the base 110, or alternatively, may be fixed using an adhesive or the like.

The garment 20 further includes a plurality of connecting portions 22 electrically connected to each of two or more different portions of the fixing region. In the example of FIG. 5, a region between P₁ and P₂ of the conductive fiber 124 is defined as a fixing region, for example. In this case, the fixing region between P₁ and P₂ is not connected to the insulating material 122, and is fixed to the garment 20 or the base 110. Further, the garment 20 also has two connecting portions 22 electrically connected to P₁ and P₂ of the fixing region of the conductive fiber 124.

Thus, the measurement part 32 can calculate the inductance value of the portion of the conductive fiber 124 other than the fixing region by using the inductance between the two different connecting portions 22 provided in the fixing region as a reference value. The measurement part 32 can calculate an increase or decrease in the inductance between P₂ and P₃ and between P₃ and P₄ as compared with the inductance between P₁ and P₂, for example.

Since the fixing region of the conductive fiber 124 maintains a certain length, the measurement part 32 can measure the change in the absolute value of the inductance by measuring the value of the inductance of the fixing region in advance. Further, the length of the fixing region of the conductive fiber 124 changes due to environmental changes such as changes in temperature and the like, but the measurement part 32 can cancel the influence of such environmental changes by calculating the inductance in comparison with the fixing region.

Accordingly, the measurement apparatus 30 can accurately measure the inductance of each portion of the conductive fiber 124. It should be noted that, in the present embodiment, an example in which the fixing region is a portion of the conductive fiber 124 has been described, but the present disclosure is not limited thereto. The fixing region may be provided to the garment 20 independently of the conductive fiber 124 as long as the fixing region can be used for calculating the reference value of the inductance. Further, the fixing region may be provided inside the measurement apparatus 30.

An example has been described above in which the measurement apparatus 30 according to the present embodiment is provided in the garment 20 worn by the user, and measures the user's state by using the sensor element 100 whose inductance changes in accordance with the user's state, but the present invention is not limited thereto. The measurement apparatus 30 may further use a sensor or the like whose capacitance and/or resistance changes in accordance with the user's state.

<Example in which the Garment 20 has Other Sensors>

As an example, the garment 20 further includes at least one of a) an acceleration sensor for detecting the acceleration when the user wearing the garment 20 moves and b) a gyro sensor for detecting the angular velocity and/or angular acceleration when the user wearing the garment 20 moves. In this case, the measurement apparatus 30 can measure the changes in the user's posture such as moving, rising, sitting, and lying back on the basis of the detection signal from the acceleration sensor and/or the gyro sensor. Accordingly, the measurement apparatus 30 can accurately measure the breathing, heartbeat, and the like of the user, excluding noise components caused by such a movement and the like of the user.

At least a part of the monitoring system 10 according to the present embodiment is a computer or the like, for example. The computer functions as at least a part of the measurement apparatus 30, the acquisition part 40, the storage 50, the detection part 60, and the notification part 70 according to the present embodiment by executing a program or the like, for example.

The computer includes a processor such as a CPU, and functions as at least a part of the measurement apparatus 30, the acquisition part 40, the storage 50, the detection part 60, and the notification part 70 by executing a program stored in the storage part 34 and/or the storage 50. The computer may further include a GPU (Graphics Processing Unit) or the like.

The present invention is explained on the basis of the exemplary embodiments. The technical scope of the present invention is not limited to the scope explained in the above embodiments and it is possible to make various changes and modifications within the scope of the invention. For example, the specific embodiments of the distribution and integration of the apparatus are not limited to the above embodiments, all or part thereof, can be configured with any unit which is functionally or physically dispersed or integrated. Further, new exemplary embodiments generated by arbitrary combinations of them are included in the exemplary embodiments of the present invention. Further, effects of the new exemplary embodiments brought by the combinations also have the effects of the original exemplary embodiments.

What is claimed is:

1. A garment worn by a user, comprising:
a sensor material that includes a) an insulating material stretchable in a longitudinal direction and b) a conductive fiber having conductivity and coiled around the insulating material; and
a plurality of connecting portions electrically connected to three or more portions of the conductive fiber; wherein
at least a portion of the conductive fiber includes a fixing region fixed such that the fixing region does not stretch or shrink even when the insulating material stretches and shrinks, and
at least two connecting portions of the plurality of connecting portions are electrically connected to each of two different portions of the fixing region.

2. The garment according to claim 1, wherein
the conductive fiber is fixed to different portions of the insulating material.

3. The garment according to claim 1, further comprising:
a base formed as a string or strip using a thread that can stretch and shrink in a longitudinal direction, wherein
the sensor material is coupled to the base such that the base can be stretched and shrunk in a direction in which the sensor material stretches and shrinks.

4. The garment according to claim 1, wherein
the garment is formed using a cloth that is stretchable at least in part,
the sensor material is coupled to the stretchable cloth, and
the cloth can be stretched and shrunk in a direction in which the sensor material stretches and shrinks.

5. The garment according to claim 1, wherein
the plurality of connecting portions are portions of buttons provided to the garment.

6. A measurement apparatus for measuring a state of a user wearing a garment, wherein
the garment includes
a sensor material that includes a) an insulating material stretchable in a longitudinal direction and b) a conductive fiber having conductivity and coiled around the insulating material, and
a plurality of connecting portions electrically connected to three or more portions of the conductive fiber,
the measurement apparatus includes
a measurement part that measures impedance between two different connecting portions of the plurality of connecting portions of the garment,
a switching part that switches an electrical connection between the plurality of connecting portions and the measurement portion,
a controller that controls the switching part such that impedances between two adjacent connecting portions are measured by the measurement part, and
an identification part that identifies a state of the user wearing the garment on the basis of the measurement result of the impedance.

7. The measurement apparatus according to claim 6, wherein
at least a portion of the conductive fiber includes a fixing region fixed in a manner such that the fixing region does not stretch or shrink even when the insulating material stretches and shrinks, and
the garment further includes a plurality of the connecting portions electrically connected to each of two or more different portions of the fixing region, and
the measurement part calculates an impedance value of a portion of the conductive fiber other than the fixing region by using an impedance between two different connecting portions provided to the fixing region as a reference value.

8. The measurement apparatus according to claim 6, wherein
the measurement part measures an inductance between two different connecting portions of the plurality of connecting portions of the garment, and
the identification part identifies a state of the user wearing the garment on the basis of a result of the measurement of the inductance.

9. The measurement apparatus according to claim 6, wherein
the garment further includes at least one of a) an acceleration sensor for detecting acceleration when the user wearing the garment moves and b) a gyro sensor for detecting angular velocity and/or angular acceleration when the user wearing the garment moves.

10. A monitoring system comprising:
a garment; and
a measurement apparatus that measures a state of a user wearing the garment, wherein
the monitoring system monitors a state of the user,
the garment includes
a sensor material that includes a) an insulating al stretchable in a longitudinal direction and b) a conductive fiber having conductivity and coiled around the insulating material, and
a plurality of connecting portions electrically connected to three or more portions of the conductive fiber,
the measurement apparatus includes
a measurement part that measures impedance between two different connecting portions of the plurality of connecting portions of the garment,
a switching part that switches an electrical connection between the plurality of connecting portions and the measurement portion,
a controller that controls the switching part, such that impedances between two adjacent connecting portions are measured by the measurement part, and
an identification part that identifies a state of the user wearing the garment on the basis of the measurement result of the impedance, wherein
the monitoring system further comprising:
an acquisition part that is connected to each of the plurality of measurement apparatuses and acquires states of the plurality of users respectively wearing the plurality of garments; and
a detection part that detects an abnormal state among the states of the plurality of users.

11. The monitoring system according to claim 10, wherein
the measurement part measures an inductance between two different connecting portions of the plurality of connecting portions of the garment, and
the identification part identifies a state of the user wearing the garment on the basis of a result of the measurement of the inductance.
